(19) United States
(12) Patent Application Publication
(10) Pub. No.: US 2006/0254944 A1
(43) Pub. Date: Nov. 16, 2006

(54) Dehydration and preservation method for a sanitary utensil containing bio-specimen and the storage device thereof

(76) Inventors: Yi-Feng Kao, Sanchong City (TW); Ching-Yu Lin, Yilan City (TW); Peter Chan, Taipei (TW); Yi-Jiue Tsai, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/125,094

(22) Filed: May 10, 2005

(51) Int. Cl. A01N 1/02; A61B 19/02

(52) U.S. Cl. 206/438; 435/2

(57) ABSTRACT

The present invention discloses a dehydration and preservation method for a sanitary utensil containing bio-specimen. First, a closed system for storing the sanitary utensil containing bio-specimen is provided. Next, a drying agent is provided in the closed system to remove the water vapor evolved from the bio-specimen. Additionally, a deodorization agent can be provided in the closed system to absorb the smell of the bio-specimen. Moreover, this invention also discloses several storage devices for storing a sanitary utensil containing bio-specimen.

### BACKGROUND OF THE INVENTION

1. Field of the Invention

The present invention is generally related to a dehydration and preservation method for a specimen, and more particularly to a dehydration and preservation method for a sanitary utensil containing bio-specimen and the storage device thereof.

2. Description of the Prior Art

The goal of laboratory medicine is to analyze various components of specimens to receive enough information about the physiological function, state and infections of human body, wherein several analysis is adopted for different specimens, such as: serologic analysis, immunoassay, biochemical analysis, analysis of disease genes, pharmacogenetic polymorphism analysis, analysis of hormone, and so on. Some common bio-specimens include: stool, urine, blood, sample of cervix, sample of throat culture, and they can be divided into three groups according to their characteristics:
(1) solid specimens, such as stool, hair, tissue . . . ;
(2) semisolid specimens, such as sample of cervix, sample of throat culture . . . ;
(3) non-solid specimens, such as urine, blood . . .

Traditionally, collection and storage techniques are varied with different kinds of specimen. For example, blood specimen are usually stored in test tube and refrigerated at 2-8 degrees for transportation; sputum, urine and stool specimen are usually stored in sterile container for transportation; sample of cervix is collected by inserting a speculum into examinee's vagina and holding open the walls of the vagina so that the doctor can use special swabs to scrape some cells from the surface and the inside of the cervix. These cells are then smeared onto glass slides, sprayed with a preservative to protect the cells, and then sent to a laboratory for examination under a microscope. Moreover, the used swabs are stored with preservative in a closed container for further analysis.

Additionally, the traditional skills about collection, storage and transportation of DNA specimens are remained crude. Common DNA specimens, include blood, semen and sputum, are generally dried in an open space at atmosphere environment for a long time, which is not sanitary and can not keep the bio-specimens from pollutions. To solve the above-mentioned problems associated with the dehydration and preservation method for bio-specimens, a new method and storage device are still needed corresponding to improve the quality of bio-specimens and increase the preservation time.

### SUMMARY OF THE INVENTION

In accordance with the present invention, a new dehydration and preservation method for a sanitary utensil containing bio-specimen and the storage device thereof are provided that substantially overcomes the drawbacks of the above problems mentioned from the conventional system.

One object of the present invention is to employ drying agents to decrease the water content of bio-specimens to inhibit the growth of bacteria or fungus, or reduce activity of enzymes within the bio-specimen, so as to prevent pollutions of bio-specimen and decrease the smell concentration of the bio-specimen.

Another object of this invention is to provide a closed system to help the drying agent for removing water vapor more efficiently, protecting the bio-specimen from pollutions, avoiding leakage of the bio-specimen or the smell thereof.

Still another object of this invention is to provide a storage device made of opaque materials (all or partially), so as to protect the bio-specimen and reduce damages caused by lights. Moreover, the provided storage device gives facilities for home-based sample collection, sample storage, and renders users conveniently to mail or transport samples to medical laboratories for disease detecting, disease surveillance, investigation of related genes or other bio-technology tests. Therefore, this present invention does have the economic advantages for industrial applications.

Accordingly, the present invention discloses a dehydration and preservation method for a sanitary utensil containing bio-specimen. First, a closed system for storing the sanitary utensil containing bio-specimen is provided. Next, a drying agent is provided in the closed system to remove the water vapor evolved from the bio-specimen. Additionally, a deodorization agent can be provided in the closed system to absorb the smell of the bio-specimen. Moreover, this invention also discloses several storage devices for storing a sanitary utensil containing bio-specimen.

### BRIEF DESCRIPTION OF THE DRAWINGS

FIG. 1 is a schematic diagram of a storage device, comprising a bag having an open-end, at least one zip, and a drying agent, in accordance with the second embodiment of the present invention;

FIG. 2A is a schematic diagram of a storage device, comprising a cap, a box, and a drying agent, in accordance with a preferred example of the third embodiment of the present invention;

FIG. 2B is a schematic diagram of a storage device, comprising a cap, a box, and a drying agent, in accordance with another preferred example of the third embodiment of the present invention;

FIG. 3A is a schematic diagram of sanitary utensil and the first casing, in accordance with a preferred example of the fifth embodiment of the present invention;

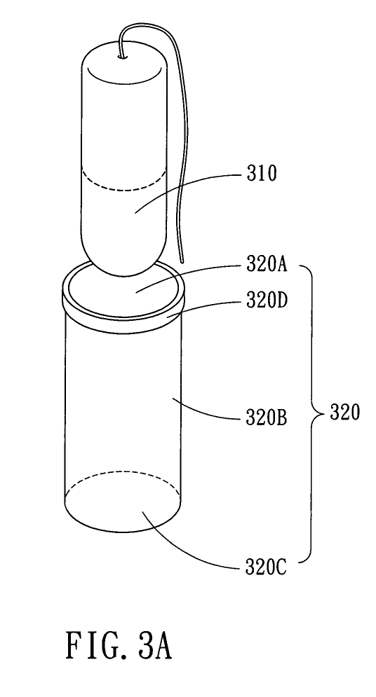

FIG. 3B is a schematic diagram of a first casing, a second casing and a cap, in accordance with a preferred example of the fifth embodiment of the present invention; and

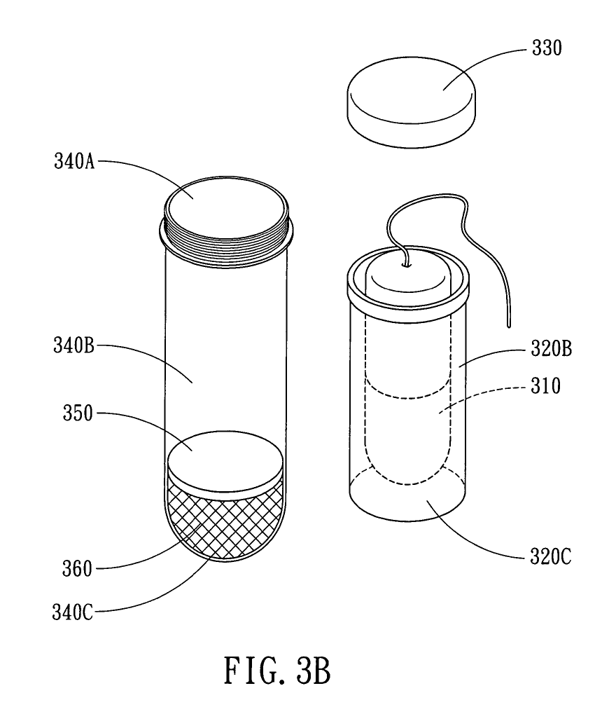

FIG. 4 is a schematic diagram of a base, a hollow shell and a drying agent, in accordance with a preferred example of the sixth embodiment of the present invention.

### DESCRIPTION OF THE PREFERRED EMBODIMENTS

What is probed into the invention is dehydration and preservation method for a sanitary utensil containing bio-specimen and the storage device thereof. Detailed descriptions of the production, structure and elements will be provided in the following in order to make the invention thoroughly understood. Obviously, the application of the invention is not confined to specific details familiar to those who are skilled in the art. On the other hand, the common elements and procedures that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In a first preferred embodiment of this invention, a dehydration and preservation method for a sanitary utensil containing bio-specimen is provided. First of all, a closed system for storing a sanitary utensil containing bio-specimen is provided. Next, a drying agent is provided in the closed system for removing the water vapor evolved from the sanitary utensil containing bio-specimen, wherein the drying agent is selected as any one or any combination of the following: (1) Compounds that react with water, such as: calcium chloride, calcium oxide, or phosphoric anhydride; and (2) Materials that absorb water, such as: silica gel, cane fiber, active carbon, or montmorillonite. Furthermore, the bio-specimen is selected as any one or any combination of the following: blood, menstrual blood, cervicovaginal secretion, urethral secretion, venereal secretion, cells and tissues shed from reproductive tract and gastral secretion. Moreover, the sanitary utensil is one of the following: swab, gauze, panty shield, pad, tampon, pantyliner, diaper, toilet paper, or other absorbent. On the other hand, a deodorization agent is further provided in the closed system for absorbing the smell of the sanitary utensil containing bio-specimen, wherein the deodorization agent is selected as any one or any combination of the following: (1) Compounds that react with the smell molecule from the sanitary utensil containing bio-specimen, such as: enzyme, zinc oxide or alkali zinc salt; and (2) Materials that absorb the smell molecule the sanitary utensil containing bio-specimen, such as: active carbon, or diatomite.

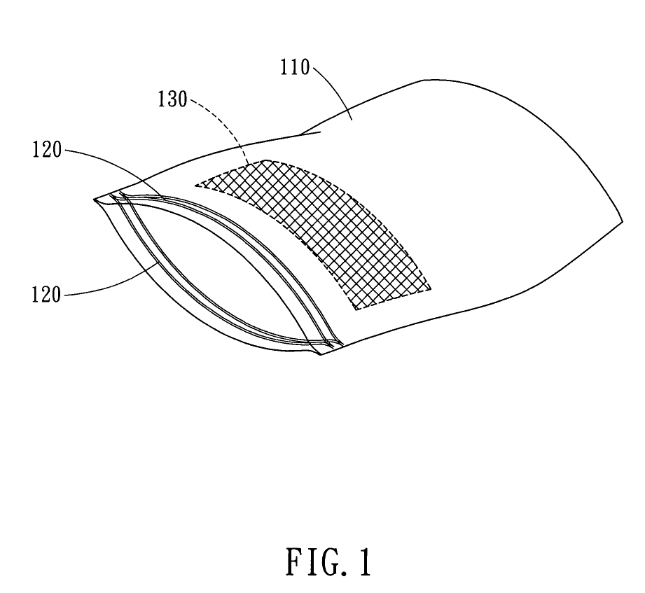

As shown in FIG. 1, in a second preferred embodiment of this invention, a storage device is provided for storing a sanitary utensil containing bio-specimen, which comprises a bag 110 having an open-end, at least one zip, and a drying agent 130 located inside the bag. The drying agent 130 is used for removing the water vapor evolved from the sanitary utensil containing bio-specimen. The zip(s) located near said open-end is to seal the storage device. According to FIG. 1, in a preferred example of this embodiment, two zips 120 are provided to seal the storage device more tightly. Further, the drying agent is located inside the bag and kept away from the bio-specimen. The mentioned drying agent is described in the first embodiment. Moreover, the storage device further comprises a deodorization agent for absorbing the smell of the sanitary utensil containing bio-specimen, wherein the deodorization agent is also described in the first embodiment, and the deodorization agent can be mixed with the drying agent. Additionally, the material of the bag is opaque, so as to protect the bio-specimen from damages caused by lights.

In a third preferred embodiment of this invention, a storage device is provided for storing a sanitary utensil containing bio-specimen, which comprises a cap, a box, and a drying agent. The box comprises a storage space for storing the sanitary utensil containing the bio-specimen, and by combining the cap and the box, the storage space can be closed. Further, the drying agent is located in the storage space, inside the cap or the box, and kept away from the bio-specimen. The drying agent is employed to remove the water vapor evolved from the sanitary utensil containing bio-specimen. The drying agent is described in the first embodiment. Additionally, the cap further comprises a first fixing element, and the box further comprises a second fixing element. The first fixing element is used to connect with the second fixing element to close the storage device. The connection between the first fixing element and the second fixing element is selected as one of the following: gluing, beclasping, engaging, screwing, and binding. Moreover, the storage device can further comprise a deodorization agent for absorbing the smell of the sanitary utensil containing bio-specimen, wherein the deodorization agent is also described in the first embodiment, and the deodorization agent can be mixed with the drying agent. Additionally, the material of the cap and/or the box is opaque, so as to protect the bio-specimen from damages caused by lights.

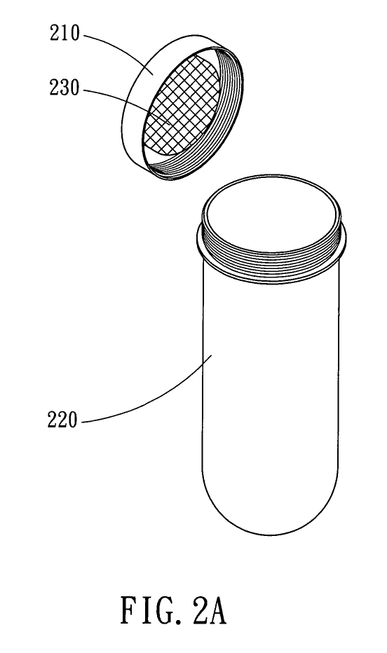

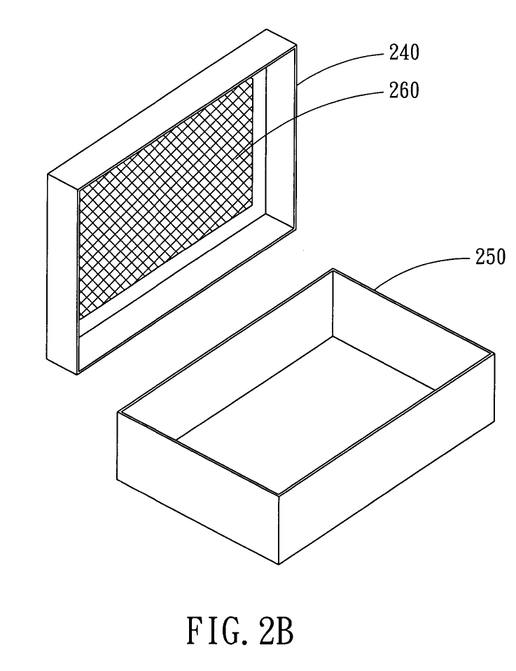

According to FIG. 2A, in a preferred example of this embodiment, a storage device is provided for storing a sanitary utensil containing bio-specimen, which comprises a first cap 210, a first box 220 and a drying agent 230. The first box 220 comprises a storage space for storing the sanitary utensil containing the bio-specimen, and by combining the first cap 210 and the first box 220, the storage space can be closed. Further, the drying agent 230 is located inside the first cap 210, and kept away from the bio-specimen. The mentioned drying agent 230 is employed to remove the water vapor evolved from the sanitary utensil containing bio-specimen. Furthermore, the first cap 210 is screwed to the first box 220. On the other hand, according to FIG. 2B, in another example of this embodiment, a storage device is provided for storing a sanitary utensil containing bio-specimen, which comprises a second cap 240, a second box 250 and a drying agent 260. The second box 250 comprises a storage space for storing the sanitary utensil containing the bio-specimen, and by combining the second cap 240 and the second box 250, the storage space can be closed. Further, the drying agent 260 is located inside the second cap 240, and kept away from the bio-specimen. The mentioned drying agent 260 is employed to remove the water vapor evolved from the sanitary utensil containing bio-specimen

In a fourth preferred embodiment of this invention, a storage device is provided for storing a sanitary utensil containing bio-specimen, which comprises a cap, a box, a drying agent and a bag. The box comprises a storage space for storing the sanitary utensil containing the bio-specimen, and by combining the cap and the box, the storage space can be closed. Further, the drying agent is located in the storage space, inside the cap or the box, and kept away from the bio-specimen. The mentioned drying agent is employed to remove the water vapor evolved from the sanitary utensil containing bio-specimen. The drying agent is described in the first embodiment. Additionally, the bag is used to store the cap, the box and the drying agent and keep them away from the surrounding. Furthermore, the cap further comprises a first fixing element, and the box further comprises a second fixing element. The first fixing element is used to connect with the second fixing element to close the storage device. The connection between the first fixing element and the second fixing element is selected as one of the following: gluing, beclasping, engaging, screwing and binding. Moreover, the storage device further comprises a deodorization agent for absorbing the smell of the sanitary utensil containing bio-specimen, wherein the deodorization agent is also described in the first embodiment, and the deodorization agent can be mixed with the drying agent. Additionally, the material of any one or and combination of the following is opaque: the cap, the box and the bag, so as to protect the bio-specimen from damages caused by lights.

In a fifth preferred embodiment of this invention, a storage device is provided for storing a sanitary utensil containing bio-specimen, which comprises a first casing, a second casing, a cap and a drying agent. The first casing comprises an open-end, a wall having pores and a first storage space, wherein the first storage space is used for storing the sanitary utensil containing the bio-specimen. The second casing comprises an open-end, a wall and a second storage space, wherein the second storage space further comprises a third storage space for storing the first casing, and the open-end of the first casing further comprises a flange, wherein the flange is used to fix the open-end of the first casing on the open-end of the second casing. Additionally, the cap is used for closing the first storage space and the second storage space by combining the cap and the second casing. Further, the drying agent is located in the second storage space, between the third storage space and the inner wall of the second casing, and the drying agent is used for removing the water vapor evolved from the sanitary utensil containing bio-specimen through the pores of the first casing. The mentioned drying agent is in the first embodiment. Moreover, the storage device further comprises a deodorization agent for absorbing the smell of the sanitary utensil containing bio-specimen, wherein the deodorization agent is also described in the first embodiment, and the deodorization agent can be mixed with the drying agent. Additionally, the material of any one or any combination of the following is opaque: the first casing, the second casing and the cap, so as to protect the bio-specimen from damages caused by lights.

According to 3A and 3B, in a preferred example of this embodiment, a storage device is provided for storing a sanitary utensil containing bio-specimen, which comprises a first casing 320, a second casing 340, a cap 330 and a drying agent 360. The first casing 320 comprises a first open-end 320A, a first storage space 320B and a first bottom wall 320C having pores, wherein the first storage space 320B is used for storing the sanitary utensil 310 containing the bio-specimen. The second casing 340 comprises a second open-end 340A, a second storage space 340B and a second bottom wall 340C, wherein the second storage space 340B further comprises a third storage space for storing the first casing 320. The first casing 320 further comprises a flange 320D, wherein the flange 320D is used to fix the first open-end 320A on the second open-end 340A. Additionally, the cap 330 is used for closing the first storage space 320B and the second storage space 340B by combining the cap 330 and the second casing 340. Furthermore, the drying agent 360 is located in the second storage space 340B, between the third storage space and the second bottom wall 340C of the second casing, and the drying agent is used for removing the water vapor evolved from the sanitary utensil containing bio-specimen through the pores of the first bottom wall 320C. Moreover, a fixing layer 350 with pores is provided between the third storage space and the drying agent 360. The fixing layer 350 is employed to confine the drying agent.

In a sixth preferred embodiment of this invention, a storage device is provided for storing a sanitary utensil containing bio-specimen, wherein the conformation of the storage device is similar to a matchbox, and the storage device comprises : a base, a hollow shell and a drying agent. The base comprises a storage space for storing the sanitary utensil containing the bio-specimen. The hollow shell is used for containing the base, and by assembling the base and the hollow shell, the storage space can be closed. Additionally, the drying agent located in the storage space for removing the water vapor evolved from the sanitary utensil containing bio-specimen. Besides, the drying agent is located inside the base, and kept away from the bio-specimen. The mentioned drying agent is described in the first embodiment. Furthermore, the base further comprises a first fixing element, and the hollow shell further comprises a second fixing element. The first fixing element is used to connect with the second fixing element to close the storage device. The connection between the first fixing element and the second fixing element is selected as one of the following: gluing, beclasping, engaging, screwing and binding. Moreover, the storage device further comprises a deodorization agent for absorbing the smell of the sanitary utensil containing bio-specimen, wherein the deodorization agent is also described in the first embodiment, and the deodorization agent can be mixed with the drying agent. Additionally, the material of the base and/or the hollow shell is opaque, so as to protect the bio-specimen from damages caused by lights.

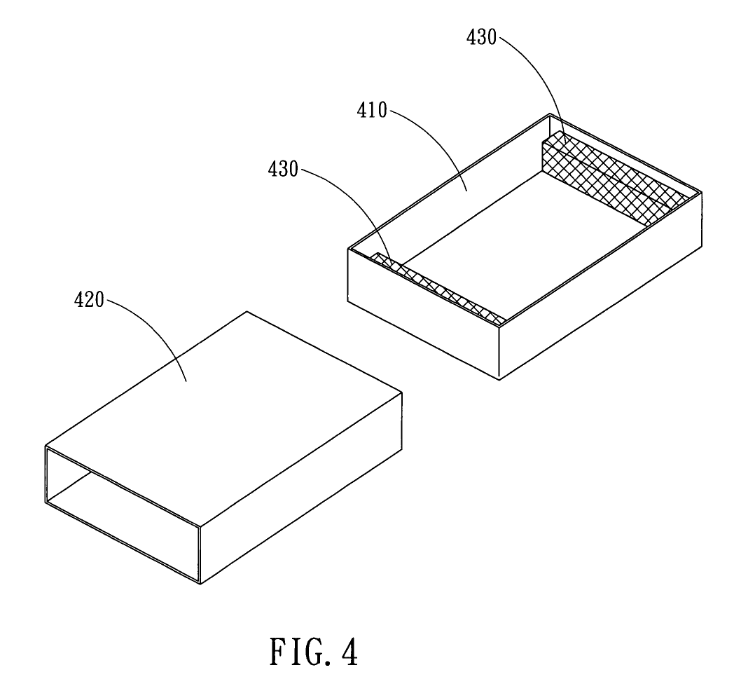

According to FIG. 4, in a preferred example of this embodiment, a storage device is provided for storing a sanitary utensil containing bio-specimen, which comprises a base 410, a hollow shell 420 and a drying agent 430. The base 410 comprises a storage space for storing the sanitary utensil containing the bio-specimen. Further, the hollow shell 420 is used for containing the base 410, and by assembling the base 410 and the hollow shell 420, the storage space can be closed. Additionally, the drying agent 430 located in the storage space for removing the water vapor evolved from the sanitary utensil containing bio-specimen. Besides, the drying agent 430 is located inside the base 410, and the drying agent 430 is kept away from the bio-specimen.

In a seventh preferred embodiment of this invention, a storage device is provided for storing a sanitary utensil containing bio-specimen, wherein the conformation of the storage device is similar to a matchbox, and the storage device comprises a base, a hollow shell, a drying agent and a bag. The base comprises a storage space for storing the sanitary utensil containing the bio-specimen. The hollow shell is used for containing the base, and by assembling the base and the hollow shell, the storage space can be closed. Further, the drying agent located in the storage space for removing the water vapor evolved from the sanitary utensil containing bio-specimen. Besides, the drying agent is located inside the base, and the drying agent is kept away from the bio-specimen. The drying agent is described in the first embodiment. Additionally, the bag is used to store the base, the hollow shell and the drying agent, and keep them away from the surrounding. The mentioned base further comprises a first fixing element, and the hollow shell further comprises a second fixing element. The first fixing element is used to connect with the second fixing element to close the storage device. The connection between the first fixing element and the second fixing element is selected as one of the following: gluing, beclasping, engaging, screwing and binding. Moreover, the storage device further comprises a deodorization agent for absorbing the smell of the sanitary utensil containing bio-specimen, wherein the deodorization agent is also described in the first embodiment, and the deodorization agent can be mixed with the drying agent. Furthermore, the material of any one or any combination of the following is opaque: the base, the hollow shell and the bag, so as to protect the bio-specimen from damages caused by lights.

In the above preferred embodiments, the present invention employs drying agents to decrease the water content of bio-specimens to inhibit the growth of bacteria or fungus, or reduce activity of enzymes within the bio-specimen, so as to prevent pollutions of bio-specimen and decrease the smell concentration of the bio-specimen. Another object of this invention is to provide a closed system to help the drying agent for removing water vapor more efficiently, protecting the bio-specimen from pollutions, avoiding leakage of the bio-specimen or the smell thereof. Still another object of this invention is to provide a storage device made of opaque materials (all or partially), so as to protect the bio-specimen and reduce damages caused by lights.

To sum up, the present invention discloses a dehydration and preservation method for a sanitary utensil containing bio-specimen. First, a closed system for storing the sanitary utensil containing bio-specimen is provided. Next, a drying agent is provided in the closed system to remove the water vapor evolved from the bio-specimen. Additionally, a deodorization agent can be provided in the closed system to absorb the smell of the bio-specimen. Moreover, this invention also discloses several storage devices for storing a sanitary utensil containing bio-specimen.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A dehydration and preservation method for a sanitary utensil containing bio-specimen, comprising:
providing a closed system for storing a sanitary utensil containing bio-specimen; and
providing a drying agent in said closed system for removing the water vapor evolved from the sanitary utensil containing bio-specimen.

2. The method according to claim 1, wherein said sanitary utensil is one of the following: swab, gauze, panty shield, pad, tampon, pantyliner, diaper, toilet paper.

3. The method according to claim 1, wherein said drying agent is selected as any one or any combination of the following:
(1) Compounds that react with water, such as: calcium chloride, calcium oxide, or phosphoric anhydride; and
(2) Materials that absorb water, such as: silica gel, cane fiber, active carbon, or montmorillonite.

4. The method according to claim 1, a deodorization agent is further provided in said closed system for absorbing the smell of the sanitary utensil containing bio-specimen.

5. The method according to claim 4, wherein said deodorization agent is selected as any one or any combination of the following:
(1) Compounds that react with the smell molecule from the sanitary utensil containing bio-specimen, such as: enzyme, zinc oxide or alkali zinc salt; and
(2) Materials that absorb the smell molecule the sanitary utensil containing bio-specimen, such as: active carbon, or diatomite.

6. A storage device for storing a sanitary utensil containing bio-specimen, comprising:
a bag having an open-end;
at least one zip located near said open-end to seal said storage device; and
a drying agent located inside said bag for removing the water vapor evolved from the sanitary utensil containing bio-specimen.

7. The device according to claim 7, wherein said drying agent is kept away from the bio-specimen.

8. The device according to claim 7, wherein the material of said bag is opaque.

9. A storage device for storing a sanitary utensil containing bio-specimen, comprising:
a cap;
a box, wherein said box comprises a storage space for storing the sanitary utensil containing the bio-specimen, and by combining said cap and said box, said storage space can be closed; and
a drying agent located in said storage space for removing the water vapor evolved from the sanitary utensil containing bio-specimen.

10. The device according to claim 10, wherein said cap further comprises a first fixing element, said box further comprises a second fixing element, and said first fixing element is used to connect with said second fixing element to close said storage device.

11. The device according to claim 10, wherein the connection between said first fixing element and said second fixing element is selected as one of the following: gluing, beclasping, engaging, screwing and binding.

12. The device according to claim 10, wherein said drying agent is located inside said cap or said box, and kept away from the bio-specimen.

13. A storage device for storing a sanitary utensil containing bio-specimen, comprising:
a cap;
a box, wherein said box comprises a storage space for storing the sanitary utensil containing the bio-specimen, and by combining said cap and said box, said storage space can be closed;
a drying agent located in said storage space for removing the water vapor evolved from the sanitary utensil containing bio-specimen; and
a bag, wherein said bag is used to store said cap, said box and said drying agent, and keep said cap, said box and said drying agent away from the surrounding.

14. A storage device for storing a sanitary utensil containing bio-specimen, comprising:
a first casing comprising an open-end, a wall having pores and a first storage space, wherein said first storage space is used for storing the sanitary utensil containing the bio-specimen;
a second casing comprising an open-end, a wall and a second storage space, wherein said second storage space further comprises a third storage space for storing said first casing, and the open-end of said first casing further comprises a flange, wherein said flange is used to fix the open-end of said first casing on the open-end of said second casing;
a cap for closing said first storage space and said second storage space by combining said cap and said second casing; and
a drying agent, wherein said drying agent is located in said second storage space, between said third storage space and the inner wall of said second casing, and said drying agent is used for removing the water vapor evolved from the sanitary utensil containing bio-specimen through the pores of said first casing.

15. A storage device for storing a sanitary utensil containing bio-specimen, wherein the conformation of the storage device is similar to a matchbox, comprising:
a base, wherein said base comprises a storage space for storing the sanitary utensil containing the bio-specimen;
a hollow shell for containing said base, and by assembling said base and said hollow shell, said storage space can be closed; and
a drying agent located in said storage space for removing the water vapor evolved from the sanitary utensil containing bio-specimen.

16. The device according to claim 16, wherein said cap further comprises a first fixing element, said box further comprises a second fixing element, and said first fixing element is used to connect with said second fixing element to close said storage device.

17. The device according to claim 16, wherein the connection between said first fixing element and said second fixing element is selected as one of the following: gluing, beclasping, engaging, screwing and binding.

18. The device according to claim 16, wherein said drying agent is located inside said base, and said drying agent is kept away from the bio-specimen.

19. A storage device for storing a sanitary utensil containing bio-specimen, the conformation of the storage device is similar to a matchbox, comprising:
a base, wherein said base comprises a storage space for storing the sanitary utensil containing the bio-specimen;
a hollow shell for containing said base, and by assembling said base and said hollow shell, said storage space can be closed; and
a drying agent located in said storage space for removing the water vapor evolved from the sanitary utensil containing bio-specimen; and
a bag, wherein said bag is used to store said base, said hollow shell and said drying agent, and keep said base, said hollow shell and said drying agent away from the surrounding.
